(19) United States
(12) Patent Application Publication
(10) Pub. No.: US 2026/0083424 A1
(43) Pub. Date: Mar. 26, 2026

(54) METHODS AND SYSTEMS FOR BOOSTING POWER TO A GANTRY OF A MEDICAL IMAGING SYSTEM

(71) Applicant: GE Precision Healthcare LLC, Waukesha, WI (US)

(72) Inventors: Saeid Mirzaei, Muskego, WI (US); Antonio Caiafa, Brookfield, WI (US)

(21) Appl. No.: 19/108,082

(22) PCT Filed: Aug. 31, 2023
(86) PCT No.: PCT/US2023/031681

§ 371 Date: Feb. 28, 2025

Related U.S. Application Data
(60) Provisional application No. 63/402,567, filed on Aug. 31, 2022.

(51) Int. Cl. A61B 6/00; A61B 6/03; A61B 6/42

(52) U.S. Cl. CPC A61B 6/56; A61B 6/035; A61B 6/4275; A61B 6/4435; A61B 6/54

(57) ABSTRACT

Various methods and systems are provided for an imaging system including a an X-ray source generating X-rays; an X-ray detector positioned opposite of the X-ray source for receiving X-rays; a gantry rotatably positioned within a gantry enclosure; a gantry motor coupled to the gantry to rotate the gantry; a power distribution unit (PDU) coupled to the gantry to provide power to the gantry; and a boost converter circuitry coupled to the PDU to maintain a voltage output of the PDU during generation of X-rays by the X-ray source. The method of providing power, via the PDU, to a gantry motor to rotate the gantry; providing power, via the PDU, to the X-ray source; and activating the boost converter circuitry to provide a voltage regulation to the gantry motor rotating the gantry via a gantry motor drive at a constant rotational speed.

### CROSS-REFERENCE TO RELATED APPLICATIONS

This is a national stage application under 35 U.S. C. § 371(c) of PCT Application No. PCT/US2023/031681, filed on Aug. 31, 2023, which priority to U.S. Provisional Application No. 63/402,567, filed on Aug. 31, 2022, the disclosures of which are incorporated herein by reference in their entirety.

### BACKGROUND

Embodiments of the subject matter disclosed herein relate to providing a voltage regulator or boost converter to a power supply of a computed tomography (CT) imaging system to maintain a constant voltage for powering a gantry motor to rotate a gantry of the CT imaging system.

A gantry of a computed tomography (CT) imaging system may receive power from a power source, such as a power distribution unit (PDU). The PDU may provide the power for the gantry motor (i.e., to rotate the gantry) and for the X-ray generator (i.e., to generate an X-ray radiation beam). The gantry motor is powered by a power supply to rotate the gantry at a desired rotational speed. As the speed of the gantry increases the “reaction” of the gantry motor (known as back emf) increases and produces a voltage at its terminals-the higher is the rotation speed the higher is the “reaction voltage”. In order to send power to the gantry motor (and keep it rotating at the proper speed) the input voltage needs to be always higher than the “reaction voltage”. When the gantry motor is rotating at maximum speed produces the maximum “reaction voltage”, when the X-ray source is activated the input voltage will sag below the “reaction voltage” of the gantry motor causing the gantry motor to slow down in an uncontrolled manner. This unwanted loss of gantry rotational speed directly affects the image quality making the data collected at maximum speed virtually unusable. When the X-ray source is activated to generate X-rays, a large current is absorbed from the power network. The large current can cause large voltage sags due to the impedance (obstacle in the current path) at the power input terminals of the gantry motor, the power available for the gantry motor is decreased, which decreases the rotational speed of the gantry to a rate below the desired rotational speed. A solution for maintaining the rotational speed of the gantry while powering and using the X-ray source is desired.

### SUMMARY

This summary introduces concepts that are described in more detail in the detailed description. It should not be used to identify essential features of the claimed subject matter, nor to limit the scope of the claimed subject matter.

In one aspect, a system includes an imaging system including an X-ray source generating X-rays; an X-ray detector positioned opposite of the X-ray source for receiving X-rays; a gantry rotatably positioned within a gantry enclosure; a gantry motor coupled to the gantry to rotate the gantry; a power distribution unit (PDU) coupled to the gantry to provide power to the gantry; and a boost converter circuitry coupled to the PDU to maintain a voltage output of the PDU during generation of X-rays by the X-ray source.

In another aspect, a method of providing power, via a PDU, to a gantry motor to rotate a gantry; providing power, via the PDU, to an X-ray source; and activating a boost converter circuitry to provide a voltage regulation to the gantry motor rotating the gantry via a gantry motor drive.

### BRIEF DESCRIPTION OF DRAWINGS

The present disclosure will be better understood from reading the following description of non-limiting embodiments, with reference to the attached drawings.

FIG. 1 is a schematic diagram of an embodiment of a computed tomography (CT) imaging system.

FIG. 2 is a block diagram of an embodiment of a CT imaging system.

FIG. 3 is a block schematic diagram of an embodiment of a prior art power circuitry configuration for powering a CT imaging system.

FIG. 4 is a detailed schematic diagram of an embodiment of the prior art power circuitry configuration of FIG. 3.

FIG. 5 is a detailed schematic diagram of the power circuitry of FIG. 4 in a ramp load condition or during a gantry motor acceleration condition.

FIG. 6 is a detailed schematic diagram of of the power circuitry of FIG. 4 in an X-ray generation condition or during an X-ray exposure condition.

FIG. 7 is a block schematic diagram of an embodiment of a power circuitry configuration for powering a CT imaging system.

FIG. 8 is a detailed schematic diagram of an embodiment of a DC-DC boost converter circuitry identified in FIG. 7.

FIG. 9 is a detailed schematic diagram of an embodiment of the power circuitry configuration of FIG. 7.

FIG. 10 is a detailed schematic diagram of the power circuitry of FIG. 9 in a ramp load condition or during a gantry motor acceleration condition.

FIG. 11 is a detailed schematic diagram of of the power circuitry of FIG. 9 in an X-ray generation condition or during an X-ray exposure condition.

FIG. 12 is a flow diagram illustrating a method for providing power to a gantry motor to rotate a gantry of a CT imaging system at a constant high speed.

### DETAILED DESCRIPTION

Embodiments of the present disclosure will now be described, by way of example, with reference to the figures.

By coupling a voltage regulator or boost converter circuitry, in the form of a boost converter printed circuit board or boost converter board in between the gantry motor and the input terminals (connecting the boost converter circuitry to the input terminals of the gantry motor drive, which is coupled to the gantry motor for rotating the gantry), the boost converter circuitry will boost (step up) the voltage during a voltage sag (occurring when the X-ray source is activated - when the X-ray source is turned on) making the voltage to the gantry motor terminals virtually constant therefore allowing for a constant maximum speed of the gantry even when the X-ray source is on. This solution enables imaging exams such as cardio exams to be performed at the maximum speed of the gantry which provide unmatched image clarity.

Adding a voltage regulator or boost converter circuitry in the CT imaging system increases the gantry speed to 0.234 seconds/revolution (256 rpm). The boost converter circuitry is a high frequency switching power supply (100 kHz) with a Silicon Carbide (SiC) metal-oxide-semiconductor field-effect transistor (MOSFET), which are new technology electronic switches.

The boost converter circuitry increases the gantry speed up to 0.23 seconds/revolution based on a Silicon Carbide (SiC) power converter to fit into a confined space and have high efficiency while passing rigorous Electromagnetic Compatibility (EMC) test standards. The boost converter circuitry includes electromagnetic interference (EMI) input and output filters, and provides a high efficiency and stable output voltage (680 VDC) under heavy X-ray exposure (up to 200 KVA) which makes a deep sag in the input voltage and/or stable output voltage under a ramp load (up to 50 A) during accelerating gantry rotational speed from 0 to 300 rpm.

The boost converter circuitry includes a soft starter circuit to limit inrush current. The soft starter circuit is turning on at first to charge a huge capacitor (680 uF) inside of a gantry motor drive for less than one second (57 msec) and after turning on the main metal-oxide-semiconductor field-effect transistor (MOSFET), it will be turned off. The boost converter circuitry is protected event if the internal gantry motor drive soft starter is not working as a failure mode.

The boost converter circuitry works mainly as voltage stabilizer (regulator): when the input voltage to the boost converter circuitry falls below a certain (prefixed) value, the boost converter circuitry will operate to step up or boost the voltage and maintain a constant voltage at its output. If the input voltage does not fall, the boost converter circuitry will simply transfer the input voltage to the output voltage. Additionally, the boost converter circuitry includes noise filter circuitry.

In a CT imaging system, the boost converter circuitry is necessary to allow a maximum gantry rotation while the X-ray source is on and generating X-rays. The boost converter circuitry functions as a DC voltage regulator to step up the input voltage to a higher constant output voltage or step down the input voltage to a lower constant output voltage.

A power pan or power subassembly is located within the gantry enclosure of the CT imaging system. The outputs of the PDU are coupled to inputs of the power subassembly. The inputs of the power subassembly are coupled to a plurality of capacitors on a first capacitor printed circuit board or capacitor board, the AC output of the capacitor board are electrically coupled to an X-ray generator, which is coupled to an X-ray source. The DC output of the capacitor board is electrically coupled to the boost converter circuitry, which includes noise filter circuitry. The output of the boost converter circuitry is electrically coupled to the gantry motor drive circuitry, which is coupled to the gantry motor for rotating the gantry.

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

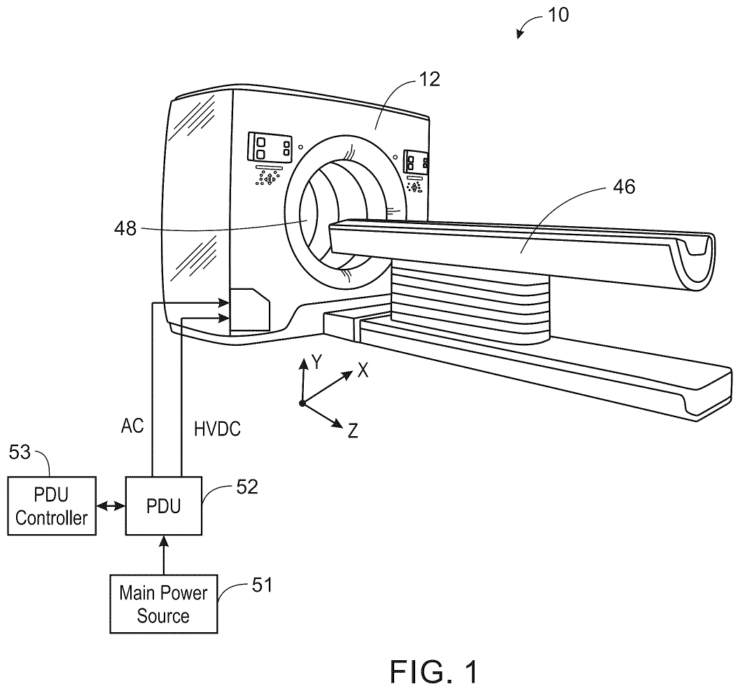

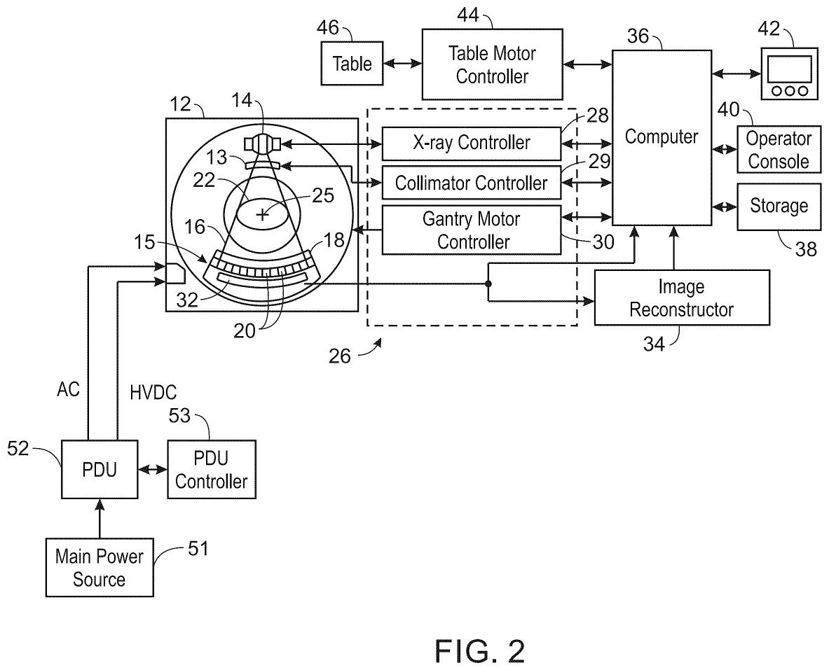

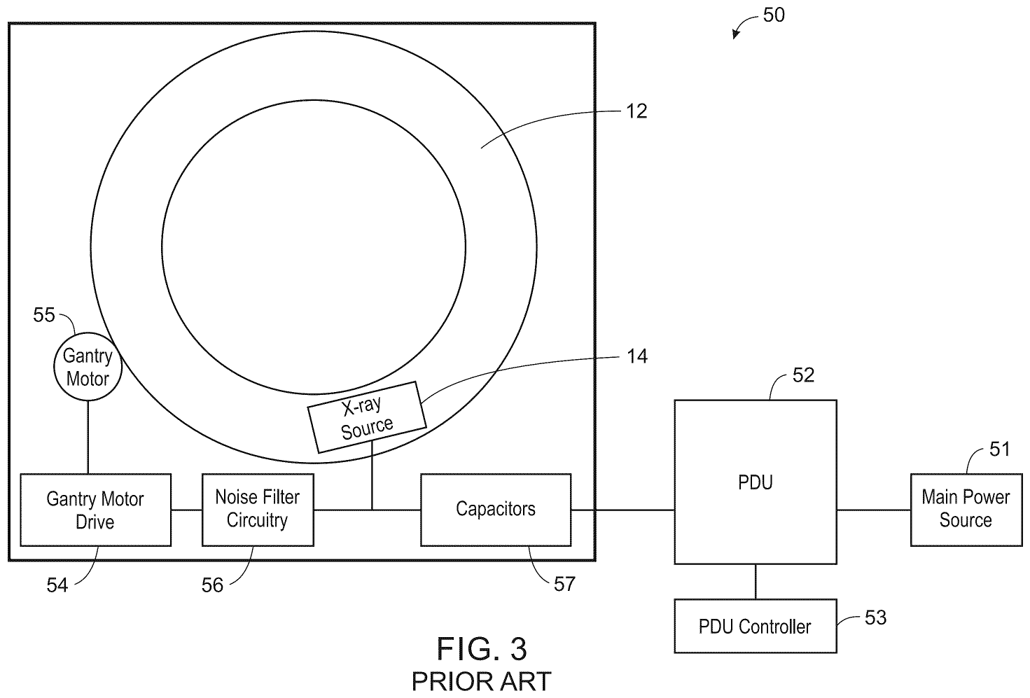

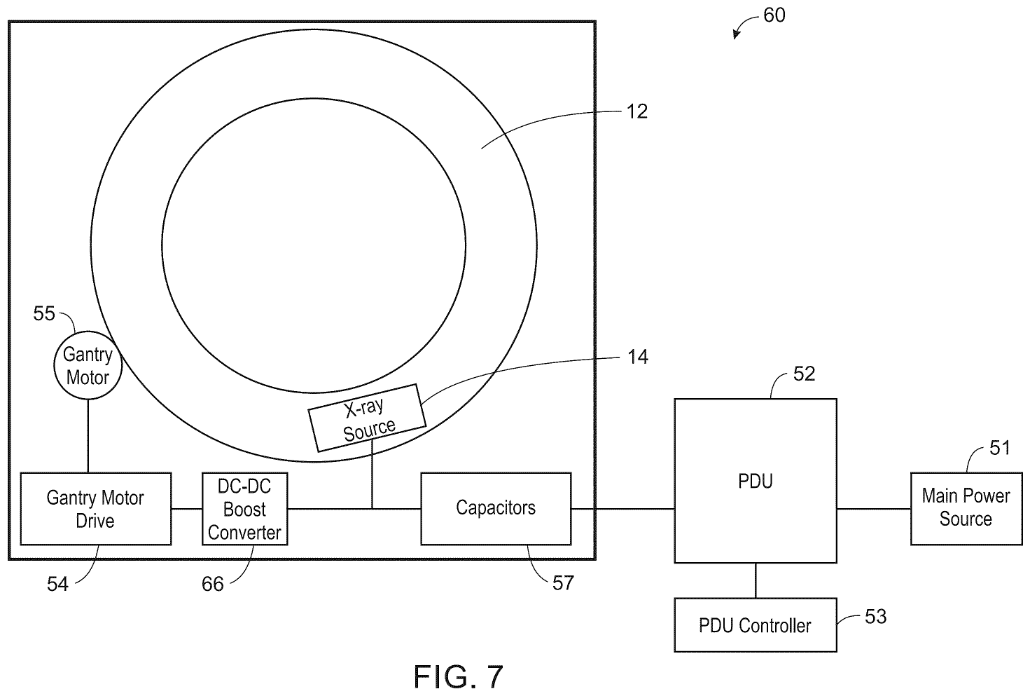

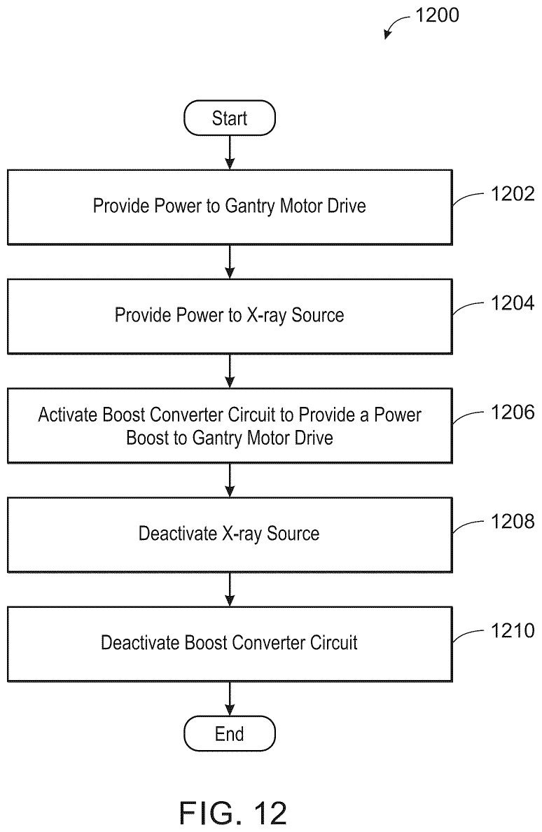

The following description relates to systems and methods that may be used to boost power provided to a gantry to maintain a constant rotational state of a gantry or maintain a rotational speed of the gantry. In particular, systems and methods are provided for a CT imaging system, but may be used with additional medical imaging systems, such as magnetic resonance imaging systems. FIGS. 1-2 show an example embodiment of an imaging system. FIGS. 3-6 depict a current power circuit of the imaging system that provides power to the gantry. FIGS. 7-11 depict an example embodiment that includes a boost converter circuitry to regulate the voltage supplied to a gantry motor, via a gantry motor drive, of the gantry to maintain a rotational speed of the gantry. FIG. 12 illustrates a method of using the example embodiment of FIGS. 7-11.

FIGS. 1 to 10 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

Though a CT imaging system is described by way of example, it should be understood that the present methods and systems may also be useful when applied to other imaging systems, such as X-ray imaging systems, magnetic resonance imaging (MRI) systems, positron emission tomography (PET) imaging systems, single-photon emission computed tomography (SPECT) imaging systems, ultrasound imaging systems, and combinations thereof (e.g., multi-modality imaging systems, such as PET/CT, PET/MR or SPECT/CT imaging systems). The present discussion of a CT imaging system is provided merely as an example of one suitable imaging system.

FIG. 1 illustrates a schematic diagram of an embodiment of a computed tomography (CT) imaging system 10 and FIG. 2 illustrates a block diagram of an embodiment of a CT imaging system. The CT imaging system includes a gantry 12. The gantry 12 has an X-ray source 14 that generates and projects a beam of X-rays 16 toward a detector assembly 15 on the opposite side of the gantry 12. The X-ray source 14 projects the beam of X-rays 16 through a pre-patient collimator assembly 13 that determines the size and shape of the beam of X-rays 16 using, for example, one or more filters. The detector assembly 15 includes a collimator assembly 18, a plurality of detector modules 20 (e.g., detector elements or sensors), and data acquisition systems (DAS) 32. The plurality of detector modules 20 detect the projected X-rays that pass through a subject or object 22 being imaged, and DAS 32 converts the data into digital signals for subsequent processing. Each detector module 20 in a conventional system produces an analog electrical signal that represents the intensity of an incident X-ray beam and hence the attenuated beam as it passes through the subject or object 22. During a scan to acquire X-ray projection data, gantry 12 and the components mounted thereon rotate about a center of rotation 25 (e.g., isocenter) so as to collect attenuation data from a plurality of view angles relative to the imaged volume.

Rotation of gantry 12 and the operation of X-ray source 14 are governed by a control system 26 of CT imaging system 10. Control system 26 includes an X-ray controller 28 that provides power and timing signals to an X-ray source 14, a collimator controller 29 that controls a length and a width of an aperture of the pre-patient collimator 13 (and, thus, the size and shape of the beam of X-rays (e.g., x-ray beam) 16), and a gantry motor controller 30 that controls the rotational speed and position of gantry 12. An image reconstructor 34 receives sampled and digitized X-ray data from DAS 32 and performs high-speed image reconstruction. The reconstructed image is applied as an input to a computer 36, which stores the image in a storage device 38. Computer 36 also receives commands and scanning parameters from an operator via console 40. An associated display 42 allows the operator to observe the reconstructed image and other data from computer 36. The operator supplied commands and parameters are used by computer 36 to provide control signals and information to DAS 32, X-ray controller 28, collimator controller 29, and gantry motor controller 30. In addition, computer 36 operates a table motor controller 44, which controls a motorized table 46 to position subject 22 and gantry 12. Particularly, table 46 moves portions of subject 22 through a gantry opening or bore 48. A main power source 51 provides power to the gantry and the gantry components (i.e., the X-ray source 14) via a power distribution unit (PDU) 52. The PDU can provide both AC and DC power to the gantry. In some examples, the main power source 51 may be a wall outlet. A PDU controller 53 is operatively coupled to the PDU 52.

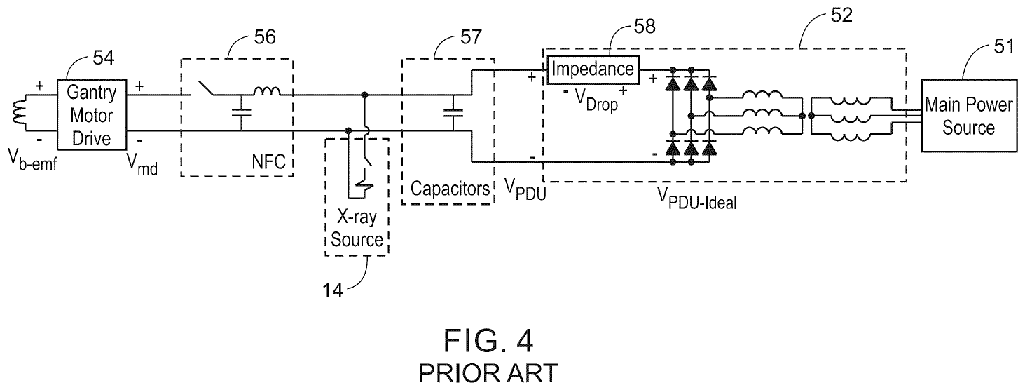

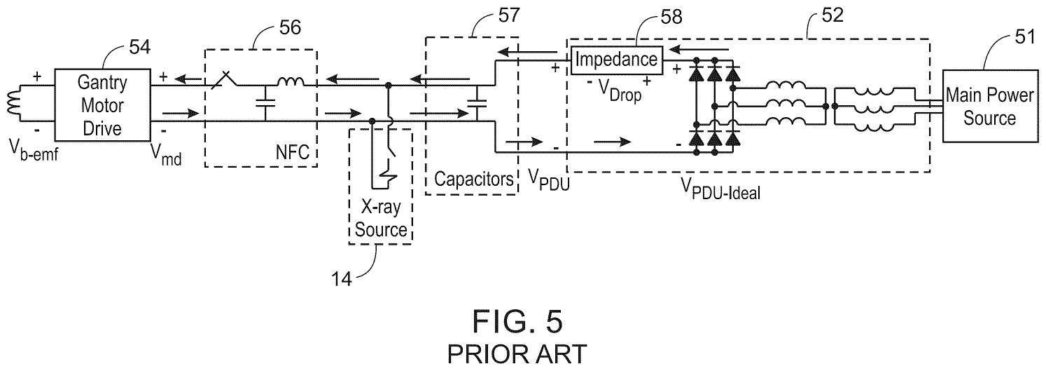

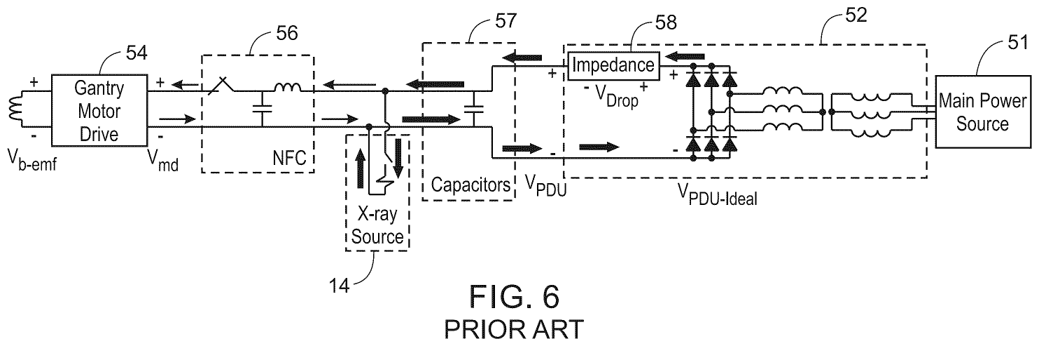

Turning now to FIG. 3, FIG. 3 illustrates a block schematic diagram of an embodiment of a prior art power circuitry 50 configuration for powering a CT imaging system 10. The power circuity 50 includes the power distribution unit (PDU) 52 to power the gantry motor drive 54 for the gantry 12 and components of the gantry 12, including the X-ray source 14, and the noise filtering circuitry (NFC) 56. Capacitors 57 may be positioned between the PDU and the gantry motor drive 54, X-ray source 14, and NFC 56. FIG. 4 illustrates a detailed schematic diagram of an embodiment of a prior art control circuitry for controlling the prior art power circuitry configuration 50 of FIG. 3. FIG. 4 shows an impedance 58 within the PDU 52 in the power circuitry 50. In particular, FIG. 4 depicts a gantry motor drive 54 and an X-ray source 14, which may draw power from the PDU 52 also shown in FIG. 3. FIG. 5 illustrates a detailed schematic diagram of the prior art control circuitry 50 of FIG. 4 in a ramp load condition or during a gantry motor acceleration condition. FIG. 5 depicts the example power circuitry 50 when power is provided via the PDU 52. The required input voltage V_{md} for the gantry motor drive to rotate the movable portion of the gantry at the desired rate (e.g., 256 rpm as example) is V_{emf} or larger, where V_{emf} is the back emf voltage created by the movable portion of the gantry while rotating, and is a function of the rotating rate. When the X-ray generator or X-ray source is not activated, the voltage from the PDU (V_{PDU}) is approximately equal to the V_{md}, which is the voltage provided to the gantry motor drive. Further, the voltage required by the gantry motor V_{emf} is less than the V_{md} in this stage. For example, power may be provided to the gantry motor drive 54 to begin rotation of the gantry 12. The PDU 52 can provide sufficient voltage to rotate the gantry 12 at the desired rotations per minute (256 rpm as example). However, when the X-ray source 14 is activated, as depicted in FIG. 6, the voltage, V_{md} supplied to the gantry motor drive to rotate the gantry 12, drops significantly because a large amount of voltage V_{Drop} drops on the impedance 58 within the PDU. The voltage drop V_{Drop} is caused by the large power that needs to be supplied to the X-ray generator during X-ray exposure. Thus, the voltage supplied to the gantry motor drive (V_{md}) is less than the voltage required by the gantry motor (V_{emf}) to keep the rotation at the desired rotations per minute. When the voltage supplied to the gantry motor drive (V_{md}) is less than the voltage required by the gantry motor (V_{emf}), the gantry 12 reduces the rpms at which the gantry 12 is rotating. This reduction in rotation speed of the gantry 12 can affect image acquisition and its quality, especially during cardiac imaging. FIG. 6 illustrates a detailed schematic diagram of of the prior art control circuitry of FIG. 4 in an X-ray generation condition or during an X-ray exposure condition.

FIG. 7 illustrates a block schematic diagram of an embodiment of a power circuitry configuration 60 for powering a CT imaging system 10. The power circuitry 60 includes a power distribution unit (PDU) 52 to power the gantry motor drive 54 for the gantry 12 and components of the gantry 12, including the X-ray source 14, and a voltage regulator or boost converter (e.g., a DC-DC boost converter circuitry) 66.

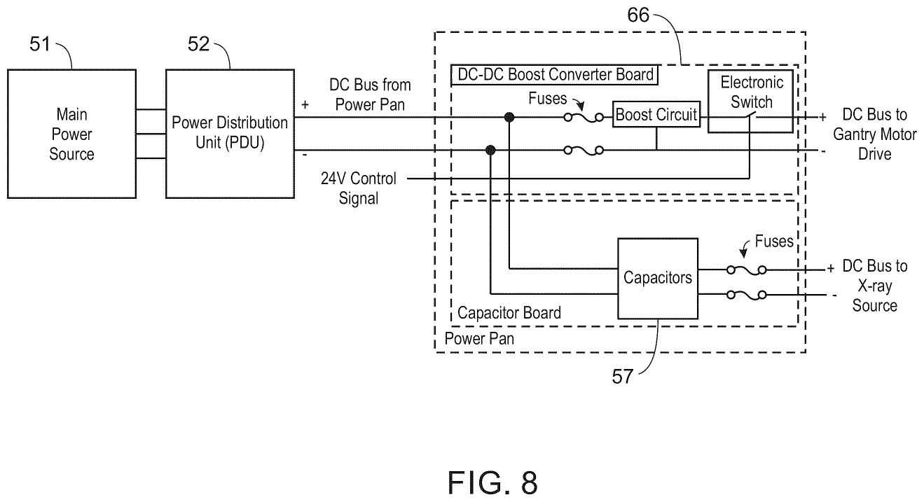

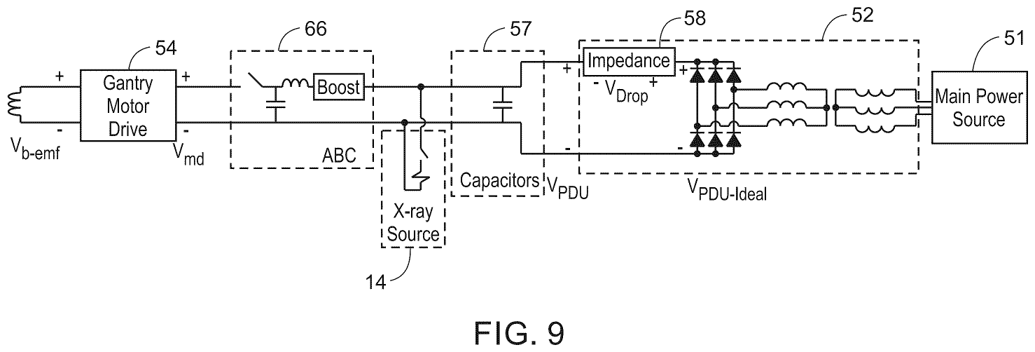

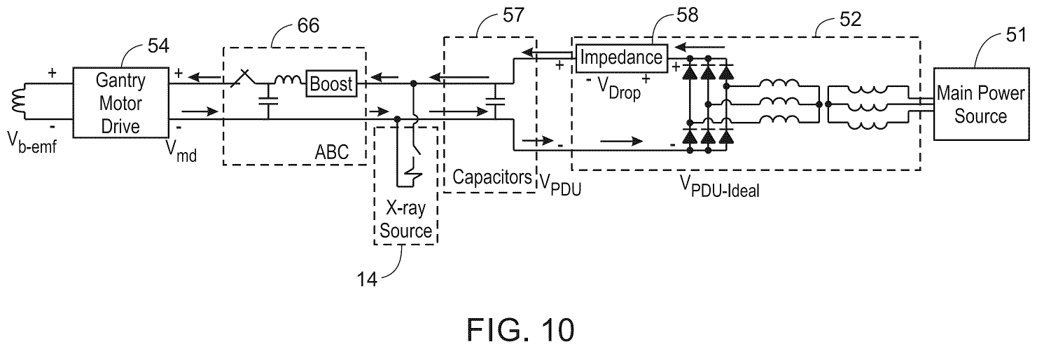

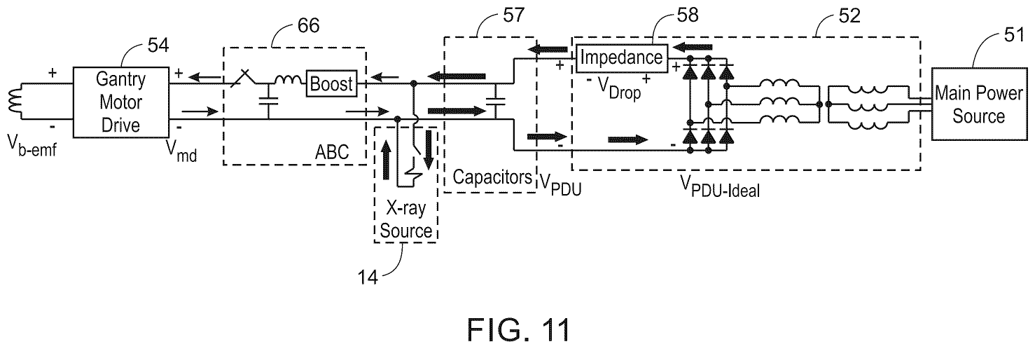

FIG. 8 illustrates a detailed schematic diagram of an embodiment of a DC-DC boost converter circuitry 66 identified in FIG. 7. FIG. 9 illustrates a more detailed schematic diagram of the power circuitry 60 configuration of FIG. 7. FIG. 10 illustrates a detailed schematic diagram of the power circuitry 60 of FIG. 9 when power is applied to the PDU 52. For example, power may be provided to the gantry motor drive 54 to begin rotation of the gantry 12. The PDU 52 can provide sufficient voltage to rotate the gantry 12 at the desired rotations per minute (256 rpm as example). When the X-ray source 14 is activated, as depicted in FIG. 11, the voltage supplied to the gantry 12 from the PDU 52 drops significantly as previously explained. FIG. 11 is a detailed schematic diagram of of the power circuitry of FIG. 9 in an X-ray generation condition or during an X-ray exposure condition. To counteract the drop in voltage, the boost converter circuitry 66 is engaged to provide a voltage regulation and keep the voltage V_{md} to the gantry motor drive 54 at its nominal value. Thus, with the boost converter circuitry 66, there is a constant voltage output from the converter to the gantry motor drive 54 for a full duration of a scan. With the boost converter circuitry 66 engaged, the voltage provided to the gantry motor (V_{md}) is still larger than the voltage required (V_{emf}), so the gantry motor is able to maintain the gantry at the desired rotational speed. The voltage boost to the gantry motor drive 54 is sufficient to keep the gantry speed to 0.234 sec/rev during image acquisition, especially during Cardiac Imaging. That is, the gantry maintains a rotational speed of the gantry 12 at the desired rpm (256 rpm as example), even during the image acquisition stage.

The use of high performance devices such as Silicon Carbide (SiC) MOSFET or Gallium Nitride (GaN) MOSFET and high frequency (such as 100 kHz or larger) operation, allow the boost converter circuitry 66 to have high power density (therefore presenting small and compact size). The small footprint enables the boost converter circuitry 66 to be positioned within an existing space of the non-rotating portion of the ganty without significantly affecting the placement and design of other components positioned within the same portion of the gantry. In some examples, the boost converter circuitry 66 includes and replaces the function of the NFC 56 within the non-rotational portion of the gantry.

The boost converter circuitry 66 is operable within a high power ranged, up to 34 kW at 100 kHz. The boost converter circuitry 66 can produce a stable output voltage of 680 VDC under heavy X-ray exposure, up to 200kVA. The boost converter circuitry 66 maintains a stable voltage output during a ramp load of up to 50 Amps during acceleration of the gantry from 0 to the desired rpm (256 rpm as example) and/or during initialization of the X-ray generator. The boost converter circuitry 66 maintains a stable voltage output if the utility power is in low line condition up to-13% of nominate voltage. The boost converter circuitry 66 can bypass the input voltage to the output stably if the utility power is in High line condition up to +10%. The boost converter circuitry 66 includes an electronic circuit used to turn on and turn off at specific threshold voltages. In some examples, the boost converter circuitry 66 can turn on if the input voltage is more than 527 VDC and turn off at lower than 427 VDC.

The boost converter circuitry 66 includes overheat protection. In some examples, the boost converter circuitry 66 will be disabled if the temperature of a heat sink exceeds a threshold temperature. The threshold temperature may be, in some examples, 90 degrees Celsius. An error may also be sent to the system log, and the boost converter circuitry will re-enable after a reset of the gantry side or HVDC.

The boost converter circuitry 66 has a specific soft starter circuit to limit inrush current. The soft starter initiates to charge a large capacitor (680 uF) inside of a gantry motor drive for less than one second (e.g., 57 msec) and after turning on a Main MOSFET, it will be turned off. The boost converter circuitry is protected event if the internal gantry motor drive soft starter is not working, as a failure mode. To protect the boost converter circuitry components, the soft starter circuit turns on when the input voltage is more than 527 VDC and turns off if the input voltage is lower than 427V.

The method begins at 1102, which includes providing power to a gantry motor to rotate a gantry 12. The method continues at 1104, which includes providing power to the X-ray generator. In order to maintain a sufficient voltage to the gantry motor, a boost circuit is activated to provide a power boost to the gantry motor at 1106. Specifically, the power boost increases the voltage supplied to the gantry motor so that the gantry motor can maintain a desired gantry rotational speed, such as 256 rpm. The boost converter circuitry is activated for the duration the X-ray generator is active. At 1108, when imaging is complete, the X-ray generator is deactivated. Finally, at 1110, the boost converter circuitry is deactivated and the method is complete.

As used herein, an element or step recited in the singular and proceeded with the word “a” or “an” should be understood as not excluding plural of said elements or steps, unless such exclusion is explicitly stated. Furthermore, references to “one embodiment” of the invention do not exclude the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein. ” Moreover, the terms “first,” “second,” and “third,” etc. are used merely as labels, and are not intended to impose numerical requirements or a particular positional order on their objects.

The control methods and routines disclosed herein may be stored as executable instructions in non-transitory memory and may be carried out by the control system including the controller in combination with the various sensors, actuators, and other engine hardware. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein, but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of the computer readable storage medium in the engine control system, where the described actions are carried out by executing the instructions in a system including the various engine hardware components in combination with the electronic controller.

Embodiments of the present disclosure shown in the drawings and described above are example embodiments only and are not intended to limit the scope of the appended claims, including any equivalents as included within the scope of the claims. It will be understood by those skilled in the art that various modifications, combinations, and changes may be made to the embodiments without departing from the present scope as defined by the appended claims. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal language of the claims.

1. An imaging system comprising:
an X-ray source generating X-rays;
an X-ray detector positioned opposite of the X-ray source for receiving X-rays;
a gantry rotatably positioned within a gantry enclosure;
a gantry motor coupled to the gantry to rotate the gantry;
a power distribution unit (PDU) coupled to the gantry to provide power to the gantry; and
a boost converter circuitry coupled to the PDU to maintain a voltage output of the PDU during generation of X-rays by the X-ray source.

2. The imaging system of claim 1, wherein the X-ray source and the X-ray detector are coupled to the gantry.

3. The imaging system of claim 1, wherein the boost converter circuitry is positioned within the gantry enclosure.

4. The imaging system of claim 1, wherein the PDU provides power to the X-ray source.

5. The imaging system of claim 3, wherein the boost converter circuitry provides voltage regulation to the gantry motor drive while the gantry is rotating and the X-ray source is powered.

6. The imaging system of claim 1, wherein the boost converter circuitry maintains a constant voltage output during a full duration of a CT scan.

7. The imaging system of claim 6, wherein the constant voltage output is 680 VDC.

8. The imaging system of claim 1, wherein the rotational speed of the gantry is constant during a CT scan.

9. The imaging system of claim 8, wherein the rotational speed of the gantry is 256 rpm.

10. The imaging system of claim 1, the boost converter circuitry may bypass the input voltage to the output stably if the utility power is in a high line condition.

11. A method of using an imaging system comprising:
providing power, via a power distribution unit (PDU), to a gantry motor to rotate a gantry;
providing power, via the PDU, to an X-ray source; and
activating a boost converter circuitry to provide a voltage regulation to the gantry motor rotating the gantry via a gantry motor drive.

12. The method of claim 11, further comprising deactivating the X-ray source.

13. The method of claim 11, further comprising deactivating the boost converter circuitry.

14. The method of claim 11, wherein the boost converter circuitry provides voltage control to the gantry motor, via the gantry motor drive, to maintain a rotational speed of the gantry.

15. The method of claim 11, wherein the rotational speed of the gantry is kept constant during operation.

16. The method of claim 15, wherein the rotational speed of the gantry is 256 rpm.
